(19) United States
(12) Patent Application Publication
(10) Pub. No.: US 2022/0183693 A1
(43) Pub. Date: Jun. 16, 2022

(54) OCCLUSIVE DEVICE WITH SELF-EXPANDING STRUTS

(71) Applicant: Shawn P. Fojtik, Park City, UT (US)

(72) Inventor: Shawn P. Fojtik, Park City, UT (US)

(21) Appl. No.: 17/686,359

(22) Filed: Mar. 3, 2022

Related U.S. Application Data
(63) Continuation-in-part of application No. 17/463,504, filed on Aug. 31, 2021.
(60) Provisional application No. 63/072,926, filed on Aug. 31, 2020.

(51) Int. Cl. A61B 17/12

(52) U.S. Cl. CPC A61B 17/1215; A61B 2017/00004; A61B 2017/1205

(57) ABSTRACT

A self-expanding occlusive device includes a hypotube. An outer diameter of the occlusive device self-expands, or increases, and the occlusive device automatically assumes its final shape upon deployment to a target location. The occlusive device may retain its increased outer diameter and final shape as it remains exposed to one or more conditions at the target location.

### CROSS-REFERENCE TO RELATED APPLICATION

This application is a continuation-in-part of U.S. application Ser. No. 17/463,504, filed on Aug. 31, 2021 and titled OCCLUSIVE DEVICE WITH SELF-EXPANDING STRUTS (“the '504 Application”). In the '504 Application, a claim for priority was made to the Aug. 31, 2020 filing date of U.S. Provisional Patent Application No. 63/072,926, titled OCCLUSIVE DEVICE WITH MULTIPLE SELF-EXPANDING STRUTS, SHAPES, and METHODS (“the '926 Provisional Application”) pursuant to 35 U.S.C. § 119(e). The entire disclosures of the '926 Provisional Application and the '504 Application are hereby incorporated herein.

### TECHNICAL FIELD

This disclosure relates generally to self-expanding devices that occlude voids and passages (e.g., arteries, veins, other vessels, chambers, and other like structures) within a body of a subject. More specifically, this disclosure relates to self-expanding occlusive devices formed from hypotubes. Methods of occluding voids and passages within a subject's body are also disclosed, as are methods of manufacture.

### RELATED ART

Occlusive devices, including coils and plugs, are used to therapeutically and diagnostically slow or stop blood flow and occlude other voids within a subject's body. FIGS. 1A and 1B, which are images of the same vasculature, respectively, before and after placement of occlusive devices, show the effects occlusive devices on the vasculature of a subject. Occlusive devices may be used for a variety of purposes, including the treatment of arteriovenous malformations, bleeds, perforations, aneurysms, tumors (e.g., devascularization, etc.), varices, congestion, and other conditions.

Occlusive devices, such as coils, are typically self-expanding devices designed to be constrained in a loading device, then pushed through tubular catheter, sheath, needle, cannula (each, a “delivery device”), or other like device to a target location(s), and then exit the tip of the delivery device and self-expand to promote therapeutic occlusion. Metal based coils are more common than polymer coils. Certain coils include polymer, fibers, coatings, fabrics, marker bands, and other features on outside of the metal or polymer scaffold, between scaffold features, and/or proximal or distal to the scaffold features.

FIGS. 2A and 2B, taken from White, Ken, Cloft, and Kallmes, “Coils in a Nutshell: A Review of Coil Physical Properties,” AJNR, August 2008 (“White”), illustrate a specific design for an occlusive device that comprises a coil. The coil shown in FIGS. 2A and 2B includes a thin solid wire 1° (primary structure, or “primary wire”) with a wire diameter D1. The thin solid wire 1° is shaped into a coiled wire 2° (secondary structure, or “secondary spring” and/or “primary wind.”) with a coiled wire diameter D2. The coiled wire 2° is shaped into a larger three-dimensional structure 3° (tertiary structure), such as a coiled tube, as shown in FIG. 2B, with an expandable diameter D3.

The coiled wire diameter D2 or, more specifically, the outer diameter (OD) of the coiled wire 2° defines the catheter delivery size of the coil. As an example, a coil designed for an 0.018″ delivery catheter has a coiled wire diameter D2 of ˜0.018″ OD, a coil designed for an 0.035″ delivery system typically has a coiled wire diameter D2 of ˜0.035″ OD. Manufacturers typically list their product offering under broad headings like “0.018 coils,” “0.035 coils,” and other sizes, in reference to the coiled wire diameters D2 of their coils.

As an alternative to a coiled wire 2°, a solid wire or a solid composite wire may be used for the coiled tube 3°.

The coiled tube 3° represents the final expanded and unconstrained OD, or tertiary shape, of the coil. As an example, a “035 5 mm×2 cm coil” has a coiled wire diameter D2 of 0.035 inch, a 5 mm unconstrained expandable diameter D3, and a length of 2 cm. In clinical use, there is variation between manufacturers on how to size coils for placement in target anatomy. For example, Ruby (Penumbra) and Azur CX (Terumo) coils should not be oversized relative to the anatomy—a 5 mm expandable diameter D3 coil should be put into a 5 mm inner diameter (ID) vessel. However, Boston Scientific and Medtronic recommend clinicians oversize their Interlock and Concerto coils 10-20% so a 5.5 or 6 mm expandable diameter D3) coil should be put in a 5 mm ID vessel. Certain coils are recommended to be 30-50% oversized.

Occlusive devices, including coil-shaped occlusive devices, may be manufactured to form any of a number of different tertiary shapes when deployed, such as the coiled tube 3° shape, or symmetrical helix shape, depicted by FIGS. 2A and 3A, as well as a variety of other shapes, including the asymmetrical helix shape shown in FIG. 3B, the funnel shapes shown in FIGS. 3C and 3D, the ball shape shown in FIG. 3E, and a variety of other shapes. These and other shapes are referred in the art as coils.

The tertiary shape of an occlusive device, such as a coil, may enable it to perform a particular function, such as primary occlusion, framing, filling, packing, or another occlusive function. Packing and filling coils may be used inside or adjacent to (e.g., behind, etc.) a coil that provides a primary occlusion. Packing and filling coils may also be used within voids (e.g., aneurysm sacs, etc.), as shown in FIG. 4. Framing coils may frame a target like the neck of void (e.g., the neck of an aneurysm, etc.) to corral packing and/or filling coils within the void or to corral embolic material inside the void.

While existing occlusive devices are useful, the occlusion they provide is limited by the extent to which their basic structures and any coatings or ancillary materials on their basic structures can be packed together as the occlusive devices assume their tertiary shapes. Occlusion of a blood vessel typically requires the placement of five or more conventional occlusive devices in proximity to each other within the blood vessel.

### SUMMARY

An occlusive device according to this disclosure comprises, consists essentially of, or consists of a self-expanding body. The body may expand in a manner that expands its outer diameter (OD) (i.e., a first degree of expansion) and enables the hypotube to assume a predetermined tertiary shape, or its desired occlusive shape or final shape (i.e., a second degree of expansion). Such an occlusive device may be referred to as a “plug.” As few as one or two plugs may be required to occlude a blood vessel.

In some embodiments, the body of the occlusive device may comprise a hypotube with an expandable section. The expandable section may include a plurality of slits that define struts. The slits may be arranged in a manner that allows the hypotube to expand from its native OD, or its unexpanded OD, to an expanded OD. The expanded OD of the occlusive device may be about twice or even about three times its native OD. The hypotube may also be shaped in a manner that ultimately enables it to expand to its predetermined final shape, or its desired occlusive shape.

The hypotube of the occlusive device may be formed from a substantially rigid material that may be constrained into a shape that facilitates its insertion into and/or removal from a body of a subject but expand upon removal of a constraining force. Without limitation, the hypotube may be made from a metal (e.g., a nitinol, a stainless steel, etc.) or a polymer (polyether ether ketone (PEEK), etc.). The hyptotube may be formed from a shape-memory material. In some embodiments, including but not limited those where the hypotube is made from a shape-memory material, the occlusive device may assume a desired shape, or its final shape, upon exposure to conditions (e.g., temperature, moisture, etc.) at an intended target location and removal of any constraining force.

In some embodiments, rows of slits may be defined along the length of an expandable section of the hypotube of the occlusive device. Each row of slits may be positioned along a generator of the expandable section (i.e., a line extending from one end of an expandable section of the hypotube to the other end of the expandable section, parallel to an axis of the expandable section). Alternatively, each row of slits may be somewhat helically oriented around the hypotube. The slits of each row being may be offset from the slits of an adjacent row. Each slit may overlap about half of one (if the slit is located at or near an end of the hypotube) or two (if the slit is intermediately located) slits of an adjacent row; stated another way, the slits of an expandable section may have a so-called “brickwork” arrangement, or they may be arranged like the bricks in a so-called “running bond pattern.” Such an arrangement of slits and the struts defined by the slits of adjacent rows may enable the expandable section of the hypotube to assume a desired final shape (e.g., a symmetrical helix, an asymmetrical helix, a funnel, a modified funnel, a sphere, or any other desired shape.

In some embodiments, the slits of the expandable section of the hypotube of the occlusive device may be arranged in a manner that enable the struts to torque and/or twist or rotate as the expandable section or a portion thereof expands. Such an arrangement may also enable an expanded portion of the expandable section to return to its an unexpanded state once an appropriate constraining force is applied to the hypotube (e.g., when an external force constrains the hypotube into a tube, the tube's diameter will decrease and the rotating struts will rotate back to a flat non-rotated position, etc.). With such an arrangement, when the expandable section in a constrained state, or an unexpanded state, it may have a smooth outer surface, which may facilitate its movement through a catheter. As the expandable section expands and its struts rotate, the struts may engage surrounding tissues (e.g., tissues of a vessel, etc.), which may hold the expandable section in place relative to the surrounding tissues.

The manufacture of an occlusive device according to this disclosure may include cutting slits at appropriate locations into the wall of a hypotube. The hypotube may be loaded onto a mandrel to form the hypotube to a desired shape. The desired shape of the hypotube and, thus, the desired shape for the occlusive device may be set (e.g., by heating when the hypotube is formed from nitinol, etc.). A constraining force may then be applied to the hypotube, or the occlusive device, to cause it to contract, or shrink. The constraining force may contract the hypotube to a shape and dimensions that facilitate its storage and its subsequent insertion into the body of a subject. In some embodiments, the occlusive device may be constrained within a loading device.

When use of the occlusive device is desired, it may be introduced into the body of a subject. For example, a catheter may be advanced to a desired location within the body of the subject. The occlusive device may be introduced into a proximal end of the catheter and advanced through a lumen of the catheter to the desired location. As the occlusive device exits a distal tip of the catheter at the desired location, it may automatically assume its increased outer diameter and intended final shape and, thus, at least partially occlude the desired location in a desired manner. A condition at the target location (e.g., a temperature, etc.) may enable the occlusive device to self-expand and automatically assume its final shape and/or may enable the occlusive device to retain its increased outer diameter and final shape as it remains exposed to one or more conditions at the target location.

Other aspects of the disclosed subject matter, as well as features and advantages of various aspects of the disclosed subject matter, should become apparent to those of ordinary skill in the art through consideration of the ensuing description, the accompanying drawings, and the appended claims.

### BRIEF DESCRIPTION OF THE DRAWINGS

In the drawings:

FIGS. 1A and 1B are images of the same vasculature before (FIG. 1A) and after (FIG. 1B) placement of occlusive devices and show the effects occlusive devices on the vasculature of a subject.

FIGS. 2A and 2B depict the features of an existing occlusive device that comprises a coil;

FIG. 3A shows an embodiment of a coiled occlusive device that comprises a symmetrical helix;

FIG. 3B shows an embodiment of a coiled occlusive device that comprises an asymmetrical helix;

FIG. 3C shows an embodiment of a coiled occlusive device that comprises a funnel;

FIG. 3D shows an embodiment of a coiled occlusive device that comprises a modified funnel;

FIG. 3E shows an embodiment of a coiled occlusive device that comprises a somewhat spherical, or ball, shape;

FIG. 4 illustrates an occlusive device within a void—an aneurysm sac;

FIGS. 5A-5D depict an embodiment of an occlusive device with a body that includes an expandable section with an expandable and contractible outer diameter (OD);

FIGS. 6A-1 and 6A-2 respectively provide end and isometric views of a conventional occlusive device in its final shape;

FIGS. 6B-1 and 6B-2 respectively provide end and isometric views of an embodiment of an occlusive device of this disclosure with its body in an unexpanded state while assuming in its final shape;

FIGS. 6C-1 and 6C-2 respectively provide end and isometric views of the embodiment of occlusive device shown in FIGS. 6B-1 and 6B-2 with its body in an expanded state and assuming its final shape;

FIG. 7B provides a detailed end view of the embodiment of the occlusive device shown in FIGS. 6B-1 and 6B-2 with its body in the unexpanded state shown in FIGS. 6B-1 and 6B-2;

FIG. 7C provides a detailed end view of the embodiment of the occlusive device shown in FIGS. 6C-1 and 6C-2 with its body in the expanded state shown in FIGS. 6C-1 and 6C-2;

FIG. 8A is an isometric view of a variation of the embodiment of the occlusive device shown in FIGS. 6C-1, 6C-2, and 7C with its body in the expanded state shown in FIGS. 6C-1, 6C-2, and 7C and at least one of its ends being tapered;

FIGS. 8B-8E are isometric views of other variations of the embodiment of the occlusive device shown in FIGS. 6C-1, 6C-2, and 7C with the bodies of the occlusive devices in the expanded state shown in FIGS. 6C-1, 6C-2, and 7C and at least one end of each occlusive device being oriented along a longitudinal axis of the expanded occlusive device;

FIGS. 8F and 8G provide a two-dimensional representation of an embodiment of a cut pattern for forming occlusive devices with somewhat spherical shapes, such as those depicted by FIGS. 9A-9E from a hypotube;

FIGS. 8H-8J variations on the cut patterns shown in FIGS. 8F and 8G;

FIGS. 9A-9C are respectively side, cross-sectional, and oblique views of another embodiment of an occlusive device according to this disclosure, which has a somewhat spherical shape;

FIGS. 9D and 9E show variations of the embodiment of the occlusive device shown in FIGS. 9A-9C, in which at least some of the cells that form as the body of the occlusive device expands are smaller than the cells that form as the body of the occlusive device shown in FIGS. 9A-9C expands;

FIGS. 9F and 9G provide a two-dimensional representation of an embodiment of a cut pattern for forming occlusive devices with somewhat spherical shapes, such as those depicted by FIGS. 9A-9E from a hypotube;

FIG. 10 shows another variation of the embodiment of the occlusive device shown in FIGS. 9A-9C, in which struts of the expandable section of the body of the occlusive device have serrated edges;

FIG. 11 depicts another final shape of an occlusive device according to this disclosure;

FIG. 12 illustrates an embodiment of an occlusive device with an expandable coating and/or fill material;

FIG. 13 illustrates an embodiment of an occlusive device that includes a fabric or a film covering;

FIG. 14 depicts an embodiment of a method for deploying an occlusive device at a target location within a body of a subject

FIGS. 15A-15D show embodiments of a delivery system and use thereof; and

FIGS. 15E-15G show variations of a control wire of the embodiment of the delivery system shown in FIGS. 15A-15D.

### DETAILED DESCRIPTION

With reference to FIGS. 5A-5D, an embodiment of an occlusive device 10 is depicted. The occlusive device 10 is shown in an unexpanded state, or a constrained state, which facilitates its introduction into and/or removal from a body of a subject. As illustrated by FIG. 5A, the occlusive device 10, while in the constrained state, may be elongated.

The occlusive device 10 comprises a body 12. The body 12 may be formed from any of a variety of suitable materials or from a combination of suitable materials. In some embodiments, the entire body 12 may be defined from or comprise a hypotube, which may be formed from a substantially rigid material, such as a metal. Examples of suitable metals include, but are not limited to, memory alloys (e.g., nitinol, etc.), cobalt chromium (CoCr), nickel chromium (NiCr or nichrome) alloys (including, without limitation, NiCr steel), stainless steel (e.g., 316L stainless steel, 316 stainless steel, etc.), and the like. Alternatively, the body 12 may be formed from a polymer. A suitable polymer may have a sufficient hardness (e.g., at least 35 Shore D, 35 Shore D to 55 Shore D, 35 Shore D to 72 Shore D, etc.). Examples of suitable polymers include, but are not limited to polyether ether ketone (PEEK), polyimide, nylon, polyether block amides (PEBA, such as that branded as PEBAX®), and extruded plastics (provided that they have a wall thickness that does not exceed the width of their struts 36, as explained below).

An expandable section 30 of the body 12 of the occlusive device 10 may be capable of expanding outward (e.g., radially outward, etc.) from an unexpanded state, as shown in FIG. 5A, to an expanded state and to a final shape. For example, in embodiments where the body 12 is formed from a shape memory material, such as a shape memory alloy, the expandable section 30 may expand to its final shape when exposed to appropriate conditions (e.g., body temperature, etc.). As another example, such as in embodiments where the body 12 is formed from a stainless steel or a polymer, the expandable section 30 may expand to its final shape upon removal of a constraining force from the body 12.

As shown in FIGS. 5B-5D, the expandable section 30 may be defined by series 34a, 34b, 34c, etc., of slits 32 that extend at least partially through a wall of the body 12. In some embodiments, each slit 32 may extend completely through the wall of the body 12, from its outer surface to its inner surface. In other embodiments, each slit 32 may extend only partially through the wall of the body 12 (e.g., from the outer surface of the wall toward the inner surface of the wall, etc.). The extent to which each slit 32 extends through the wall of the body 12 may depend, at least in part, upon the material from which the body 12 is formed.

The slits 32 (with the exception of some slits 32 located at the ends of the expandable section 30) may have the same lengths as one another. Adjacent slits 32 in a series 32a, 32b, 32c, etc., are spaced apart by solid, uncut regions of the body 12. These solid regions may be referred to as joints 38 or junctions.

Each series 34a, 34b, 34c, etc., may be defined by linearly aligned slits 32. The slits 32 and each series 34a, 34b, 34c, etc., may extend longitudinally along the body 12, with each series 34a, 34b, 34c, etc., being positioned along a generator of the expandable section 30 (i.e., a line extending from one end of the expandable section 30 to the other end of the expandable section 30, parallel to a longitudinal axis of the expandable section 30). Such an orientation may be referred to as a “straight” orientation. Alternatively, each series 34a, 34b, 34c, etc., may be helically oriented around the body 12.

The slits 32 of each series 34b, 34c, 34d, etc., may be offset relative to the slits 32 of each adjacent series 34a, 34b, 34c, 34d, 34e, etc. Each slit 32 in a series 34a, 34b, 34c, etc., may overlap about half of one (if the slit 32 is located at or near an end of the expandable section 30) or two (if the slit 32 is intermediately located along the length of the expandable section 30) circumferentially adjacent slits 32 of each adjacent series 34a, 34b, 34c, etc. Staggering of the slits 32 around the circumference of the expandable section 30 of the body 12 may provide the expandable section 30 with a so-called “brickwork” appearance, with solid portions of the body 12 between the slits 32 arranged in a so-called “running bond pattern.”

Circumferentially adjacent series 34a, 34b, 34c, etc., of slits 32 may be spaced equidistantly around the circumference of the body 12. The expandable section 30 may include an even number of series 34a, 34b, 34c, etc., of slits 32. In embodiments where an even number of circumferentially adjacent series 34a, 34b, 34c, etc., of slits 32 are spaced equidistantly around the circumference of the body 12, each slit 32 of the expandable section 30 may be staggered relative to its circumferentially adjacent slits 32. Alternatively, the distance between slits 32 of one circumferentially adjacent series 34a may differ from the distance between slits 32 of another circumferentially adjacent series 34c; thus, the number of slits 32 of one circumferentially adjacent series 34a may differ from the number of slits 32 of another circumferentially adjacent series 34c.

The solid potions of the body 12 that are located between each adjacent pair of series 34a and 34b, 34b and 34c, 34c and 34d, etc., of slits 32 comprise struts 36 of the expandable section 30. More specifically, each strut 36 may comprise a solid portion of the body 12 between adjacent series 34a and 34b, 34b and 34c, 34c and 34d, etc., of slits 32. Stated another way, each slit 32 comprises a gap between a pair of circumferentially adjacent struts 36. In embodiments where the series 34a, 34b, 34c, etc., are oriented along the longitudinal axis of the body 12, the struts may also be oriented along the longitudinal axis of the body; in embodiments where the series 34a, 34b, 34c, etc., are helically oriented around the body 12, the struts 36 may also be oriented helically, or as a spiral, around the body 12.

Staggering of the slits 32 may enable the expandable section 30 to expand. In some embodiments, as the expandable section 30 expands, the struts 36 may rotate. Such rotation may occur, for example, in embodiments where each ring of circumferentially aligned struts 36 around an expandable section 30 includes an even number of struts 36. As the slits rotate, they protrude outwardly (e.g., radially, etc.) from the circumference of the expandable section 30, which may secure the occlusive device 10 in place.

In other embodiments, the slits 32 are not staggered and the struts 36 do not rotate when the expandable section 30 is expanded. In such embodiments, the resulting occlusive device 10 may still expand to create multiple points of contact with the wall of a vessel or void within which the occlusive device 10 resides to secure the occlusive device 10 in place within the vessel or void.

The expandability provided by the slits 32 and struts 36 of the expandable section 30 of the body 12 of the occlusive device 10 enable the outer diameter (OD) of the body 12 to expand, providing a first degree of expansion. Additionally, as the OD of the body 12 expands, the body 12 may assume a predetermined tertiary shape, or a desired occlusive shape or final shape, providing a second degree of expansion.

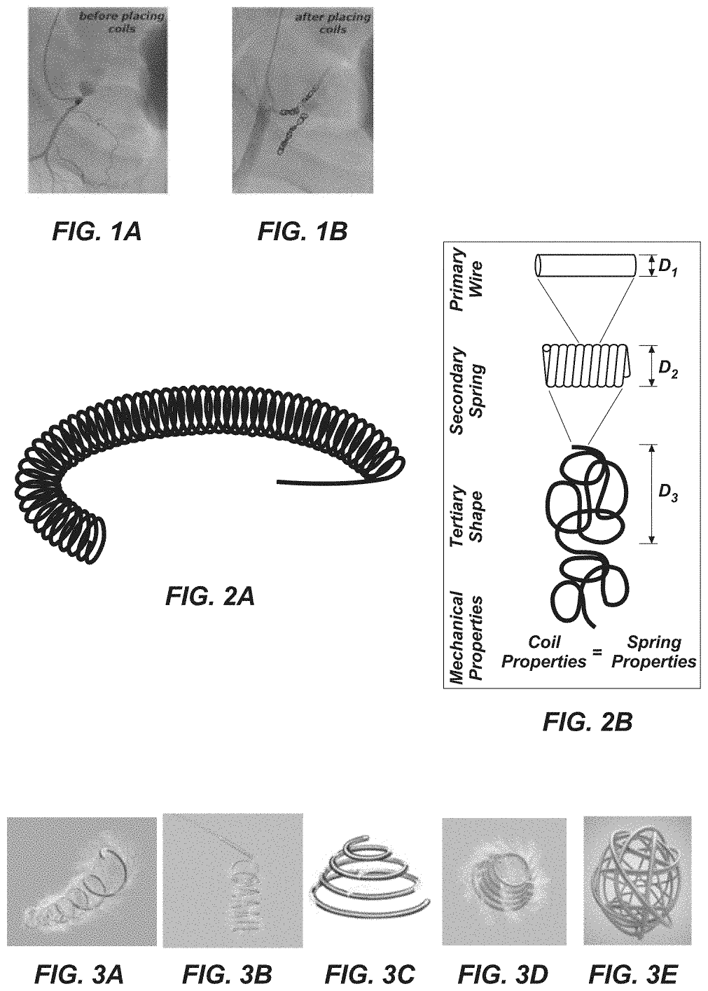

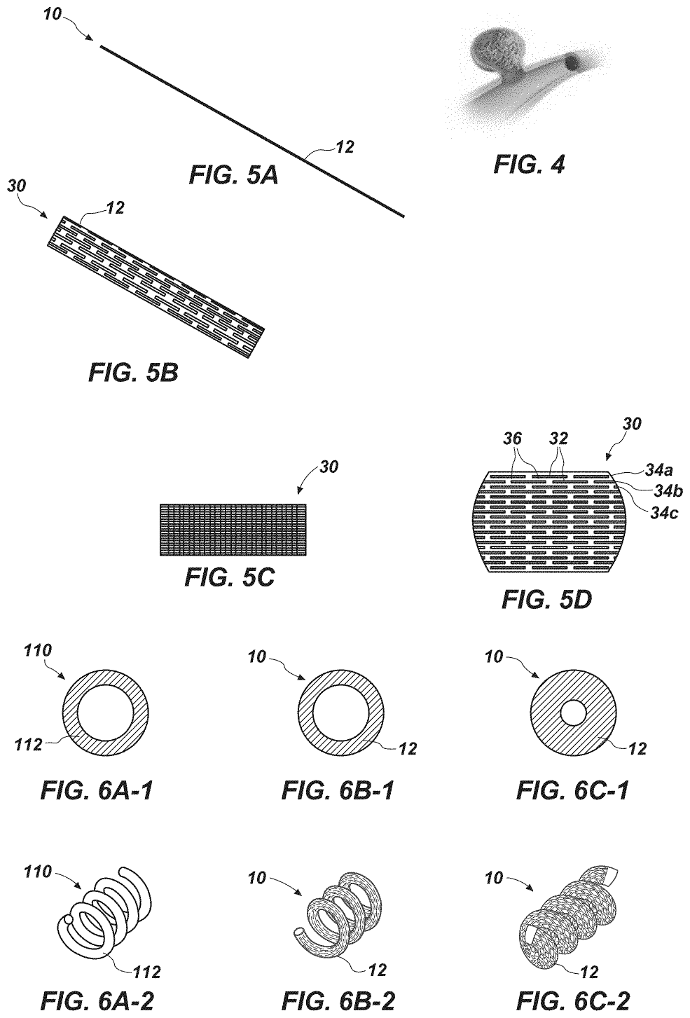

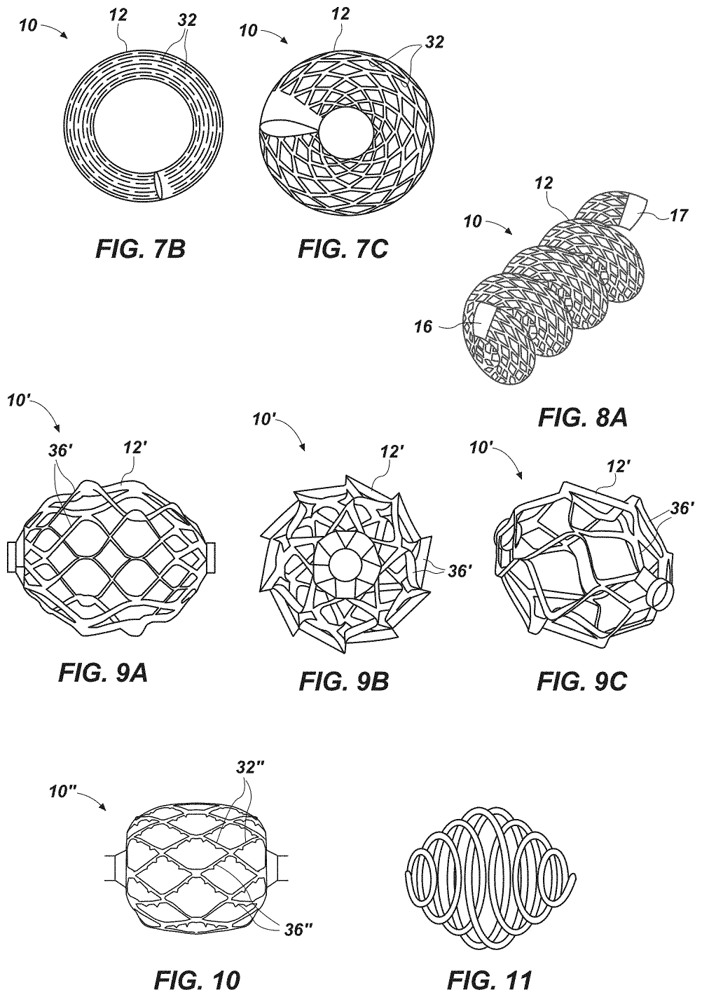

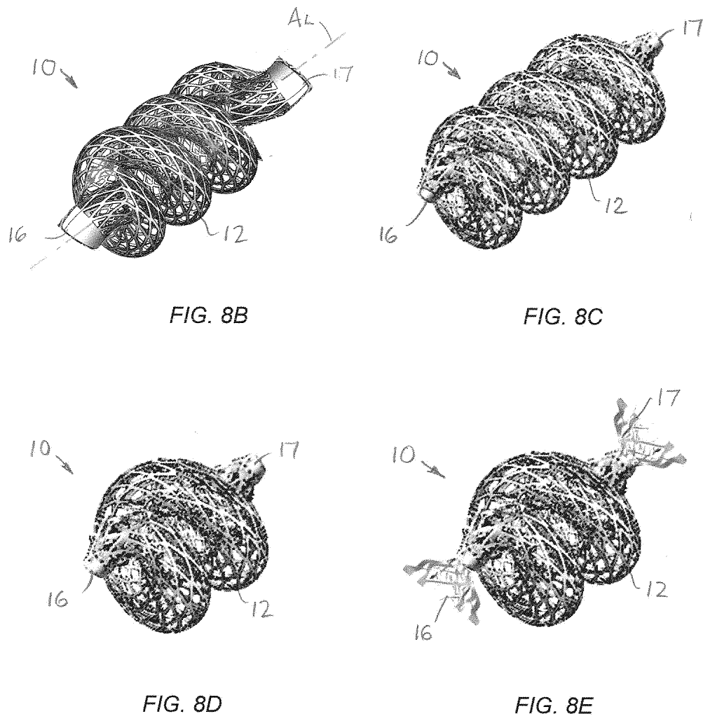

FIGS. 6A-6C contrast the occlusion provided by a single degree of expansion, as occurs when a conventional occlusive device 110 assumes its final shape (FIGS. 6A-1 and 6A-2) with the occlusion provided by two or more degrees of expansion, as occurs when an occlusive device 10 according to this disclosure expands and assumes its final shape (FIGS. 6B-1 through 6C-2). FIG. 6A-1 provides end view of an embodiment of a conventional occlusive device 10′ (e.g., a 035 5 mm×2 cm coil), which includes a coiled wire 112 that has been coiled into the final shape, also a coil, of the conventional occlusive device 110, as seen in FIG. 6A-2. Such a conventional coiled occlusive device 110 reduces an area across a lumen (e.g., a vessel, etc.) within which it is placed (e.g., by about 59%) Notably, the OD of the coiled wire 112 does not expand.

FIG. 6B-1 and 7B provide end views of an embodiment of occlusive device 10 of this disclosure with its body 12 in an unexpanded state, but assuming a coiled final shape, as seen in FIG. 6B-2 (e.g., a 035 5 mm×2 cm coil). The native dimensions of the body 12 of the occlusive device 10 (e.g., its OD, etc.) may be the same as or substantially the same as the corresponding dimensions of the coiled wire 112 of the conventional occlusive device 110 (e.g., an OD of 0.035 inch, or 0.89 mm).

While the OD of the coiled wire 112 of the conventional occlusive device 110 does not expand, as FIGS. 6C-1, 6C-2, and 7C illustrate, the OD of the body 12 of the occlusive device 10 of this disclosure can expand (e.g., to double, such as an OD of 0.070 inch, or 1.8 mm). As FIG. 7C shows, the body 12 expands as slits 32 therein open up around the circumference of the body 12. As shown in FIG. 6C-2, as the body 12 expands, the body 12 occupies an increased volume, enabling the occlusive device 10 to provide improved occlusion as it assumes its final shape (e.g., the occlusive device 10 may reduce an area across a lumen within which it is placed by at least about 75%, by at least about 80%, by at least about 85%, by at least about 90%, by about 92%).

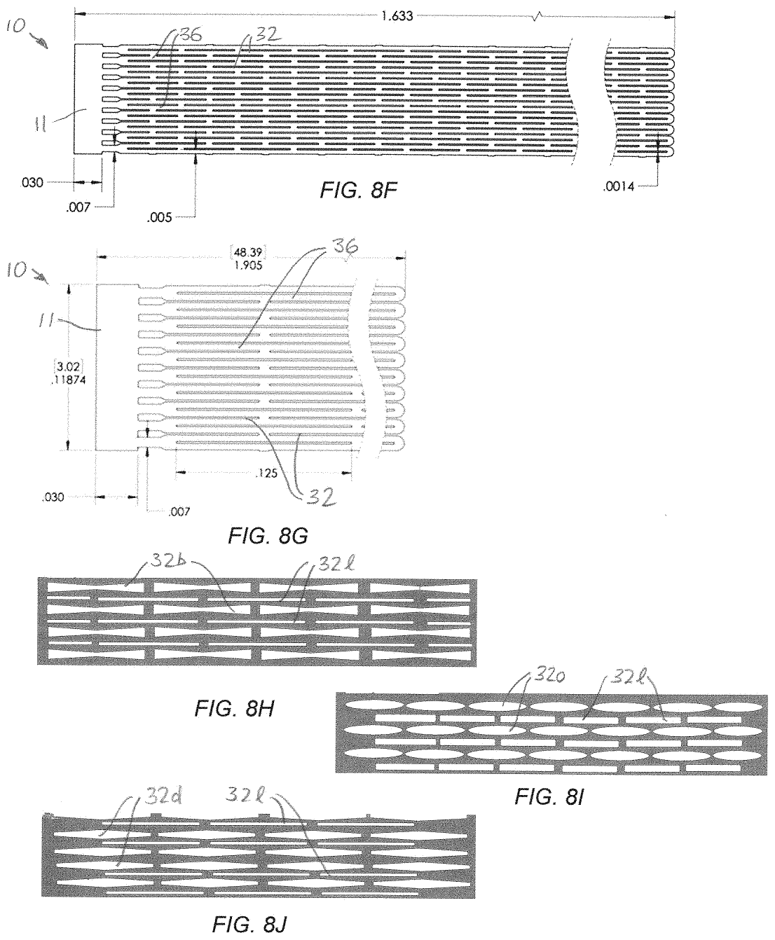

As shown in FIG. 8A, when the body 12 of the occlusive device 10 is in its expanded state, an OD of one or both ends 16, 17 of the body 12 may be the same as the OD along a remainder (central portion) of the body 12 and/or one or both ends 16, 17 may have a constricted OD (e.g., it may be tapered at its end, at a location adjacent to its end, etc.). In the depicted embodiment, end 16 has an OD that is the same as an OD of a majority of the body 12 (e.g., an OD of 0.070 inch, or 1.8 mm, etc.), while end 17 tapers to a smaller OD (e.g., an OD of 0.035 inch, or 0.89 mm, etc.).

Although FIGS. 6A-8A show coiled occlusive devices 10 with ends 16 and 17 that are oriented along the coil, the ends 16 and 17 of such an occlusive device 10 may be oriented in any of a variety of suitable directions. As an example, FIG. 8B shows a variation of the occlusive device 10 in which one or both ends 16 and 17 of the body 12 are located along a longitudinal axis AL of the coiled, expanded body 12. Such an orientation of the one of both ends 16, 17 (e.g., the forward, or distal end 16, etc.) may facilitate smooth deployment of the occlusive device 10.

While the ends 16 and 17 of the variations of occlusive devices 10 shown in FIGS. 8A and 8B have substantially the same OD as the remainder of the unexpanded body 12, the ends 16 and 17 of the variation of occlusive devices 10 shown in FIGS. 8C and 8D taper to smaller ODs than the ODs of their unexpanded bodies 12. An end 16, 17 that is tapered in this manner may be referred to as a “pinched end.”

In addition to tapering to a smaller OD that the OD of its unexpanded body 12, or being pinched, one or both ends 16, 17 of an occlusive device 10 may also flare outward, as shown in FIG. 8E. A flared end 16, 17 may expand to a same OD as the OD of the expanded body 12, or an expanded OD of a flared end 16, 17 may be smaller or larger than the OD of the expanded body of the occlusive device 10. A flared end 16, 17 may be useful in blocking blood flow, promoting clotting, anchoring the occlusive device 10 (e.g., a flared end 16, 17 may expand into the intima if the occlusive device 10 is moved in a direction the flared end 16, 17 faces, etc.), promoting endothelial tissue growth, or for any of a variety of other purposes.

FIGS. 8D and 8E show that the body 12 of a coiled occlusive device 10 may be relatively short (e.g., it may have fewer than four turns, but at least one complete turn, etc.) when compared with the bodies 12 of the longer occlusive devices 10 shown in FIGS. 8A-8C (which are depicted as having four turns, but may have more than four turns).

FIGS. 8F and 8G provide two-dimensional representations of an embodiment of a cut pattern for forming occlusive devices 10 with coiled shapes, such as that depicted by FIG. 8B, from a hypotube 11. The entire circumference of the hypotube 11 is depicted in FIGS. 8F and 8G as if it were separated, unrolled, and flattened. The cutting patterns shown in FIGS. 8F and 8G may be used to define struts 36 with widths of about 0.001 inch (about 0.025 mm) to about 0.009 inch (about 0.23 mm) and slits 32 between adjacent struts 36 with widths of about 0.001 inch (about 0.025 mm) to about 0.009 inch (about 0.23 mm) and lengths of about 0.005 inch (about 0.13 mm) to about 0.035 inch (about 0.89 mm). In some embodiments, the slits 32 may be as long as about 0.125 inch (about 3.2 mm). In embodiments where the resulting occlusive device 10 has a length of about 1.633 inches (about 41.5 mm) and an outer diameter of about 0.035 inch (about 0.89 mm) or a length of about 1.905 inch (about 48.4 mm) and an outer diameter of about 0.038 inch (about 1 mm), it may include as few as about 50 slits 32 and as many as about 500 slits 32 or more.

Although the slits 32 shown in FIGS. 8F and 8G are linear, or straight, the slits 32 and, thus, the struts 36 may have any of a variety of different shapes. As non-limiting examples, FIG. 8H shows a cut pattern with linear slits 321 and bow tie-shaped slits 32b, FIG. 8I shows a cut pattern with linear slits 321 and oval slits 32o, and FIG. 8J shows a cut pattern with linear slits 321 and diamond-shaped slits 32d. The shapes of the slits 32 may impart occlusive devices 10 with different characteristics; for example, the shapes of the slits 32 and struts 36 may affect the flexibility of the struts 36 (e.g., bow tie-shaped slits 32b may make the resulting struts 36b more flexible, etc.), the smoothness of the occlusive device 10, the expandability of the occlusive device 10, or the like.

Such an occlusive device 10 with a symmetrical helical final shape having dimensions of about 5 mm×2 cm may have 50% less metal mass than a conventional occlusive device with an OD of about 0.035 inch (about 0.89 mm) and the same final shape and dimensions. For example, a standard 035 5 mm×2 cm coil with a 0.005 inch (0.13 mm) wire has a metal volume of 2.387 mm³/cm, whereas a 035 5 mm×2 cm coil occlusive device 10 according to this disclosure formed with a hypotube having a 0.0018 inch (0.046 mm) wall thickness has a metal volume of 1.015 mm³/cm. Thus, an occlusive device 10′ of this disclosure may reduce metal volume and mass by about 50% to about 80% over the mass of a like-sized conventional occlusive device.

Reducing the metal mass will reduce the computerized axial tomography (CT) artifact of the occlusive device 10 after it has been implanted into the body of a subject. Certain subjects patients with existing conventional occlusive devices cannot be effectively imaged with CT for future follow up due to the size or location of the CT artifact produced by such conventional occlusive devices. Consequently, follow-up may require invasive angiograms. By reducing the CT artifact, subjects who receive metallic occlusive devices 10 according to this disclosure may be able to undergo CT scans for future follow up.

When expanded to its final shape, the occlusive device 10 may assume the coiled shapes depicted by FIGS. 6A-8E or any of a variety of different predetermined shapes. Such final shapes include but are not limited to the shapes shown in FIGS. 3A-3E. FIG. 11 depicts an embodiment of an occlusive device with a diamond shape, or a double-funnel shape.

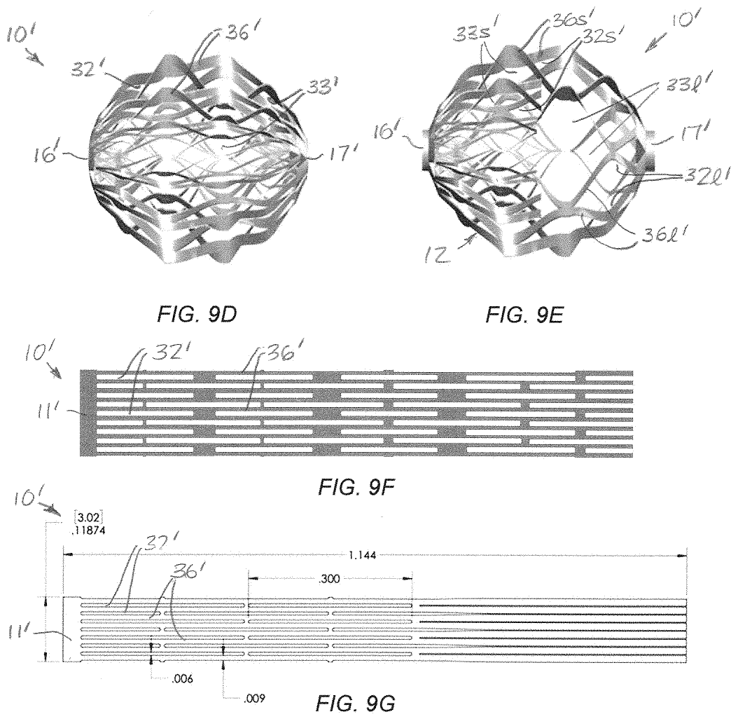

FIGS. 9A-9C provide views of an embodiment of an occlusive device 10′ that comprises a plug with a somewhat spherical (e.g., spherical, ovoid, etc.) final shape when placed in its fully expanded state (i.e., with the body 12′ of the occlusive device 10′ expanded and allowed to assume its final shape). As illustrated, when the body 12′ in its expanded state, struts 36′ may rotate outwardly (e.g., up to about 90°, etc.), which may enable the occlusive device 10′ to engage tissues (e.g., the intima of a blood vessel, etc.) against which it is positioned and expanded. Without limitation, the final shape may have a diameter of up to about 5 mm. FIG. 10 shows a variation of the occlusive device 10″ in which the slits 32″ define struts 36″ with serrated edges.

A specific embodiment of occlusive device 10′ according to this disclosure may have an unexpanded OD of about 0.035 inch (about 0.89 mm), six to eight struts 36′ around its circumference with the struts 36′ having widths of about 0.009 inch (about 0.23 mm) and slits 32′ of about 0.010 inch (about 0.25 mm) spacing each adjacent pair of struts 36′ apart from each other and lengths of about 0.035 inch (about 0.89 mm). When body 12′ of such an occlusive device 10′ expands, the struts 36′ may rotate and the slits 32′ may open up to define cells 33′ between the adjacent struts 36′. Such an occlusive device 10′ may expand from, for example, an OD of about 0.035 inch (about 0.89 mm) to an OD of about 0.105 inch (about 2.7 mm) or more.

A variation of such an occlusive device 10′ is shown in FIG. 9D and includes narrower struts 36′ and narrower slits 32′. When the body 12′ of the occlusive device 10′ expands, the dimensions of the struts 36′ and the slits 32′ may limit the distance adjacent struts 36′ may be spaced apart from each other and, thus, the sizes of the cells 33′ that form during expansion. The struts 36′ may have widths of about 0.001 inch (about 0.025 mm) to about 0.009 inch (about 0.23 mm), the slits 32′ may have widths of about 0.001 inch (about 0.025 mm) to about 0.009 inch (about 0.23 mm) and lengths of about 0.005 inch (about 0.13 mm) to about 0.030 inch (about 0.76 mm).

FIG. 9E illustrates another variation of such an occlusive device 10′, which includes struts 36s′ and slits 32s′ that define smaller cells 33s′ on one side 12s′ of its body 12′ and struts 361′ and slits 321′ that define larger cells 331′ on another side 121′ of the body 12′.

As illustrated by FIGS. 9A-9C and 9E, one or both ends 16′, 17′ of the body 12′ of the expanded occlusive device 10′ may protrude or flare somewhat. Alternatively, as shown in FIG. 9D, one or both ends 16′, 17′ of the expanded occlusive device 10′ may be flush or substantially flush with a remainder of the body 12′ of the expanded occlusive device 10′. As yet another alternative, and as also shown in FIGS. 9D and 9E, one or both ends 16′, 17′ of the expanded occlusive device 10′ may extend into the body 12′ of the expanded occlusive device 10′, or one or both of the ends 16′ and 17′ may invaginate into the body 12′.

FIGS. 9F and 9G provide two-dimensional representations of an embodiment of a cut pattern for forming occlusive devices 10′ with somewhat spherical shapes, such as those depicted by FIGS. 9A-9E, from a hypotube 11′. The entire circumference of the hypotube 11′ is depicted in FIGS. 9F and 9G as if it were separated, unrolled, and flattened. While the slits 32′ shown in FIGS. 9F and 9G are linear, or straight, the slits 32′ and, thus, the struts 36′ may have any of a variety of different shapes, as described in reference to FIGS. 8H-8J, which may impart the occlusive devices 10′ and/or their features with different characteristics (e.g., flexibility, smoothness, expandability, etc.).

As some nonlimiting examples, a somewhat spherical occlusive device 10′ that occludes a vessel with an inner diameter of about 3 mm to about 5 mm may have a length of about 6 mm to about 8 mm and an expanded outer diameter of about 6 mm. Such an occlusive device 10′ may have an unexpanded outer diameter of about 0.020 inch (about 0.5 mm) and include as few as eight struts 36′ and as many as 20 struts 36′ (e.g., 10 struts 36′, etc.) around its circumference. A somewhat spherical occlusive device 10′ that occludes a vessel with an inner diameter of about 5 mm to about 7 mm may have a length of about 8 mm to about 10 mm and an expanded outer diameter of about 8 mm. Such an occlusive device 10′ may have an unexpanded outer diameter of about 0.035 inch (about 0.89 mm) and include as few as 10 struts 36′ and as many as 40 struts 36′ (e.g., 20 struts 36′, 30 struts 36′, 40 struts 36′, etc.) around its circumference. A somewhat spherical occlusive device 10′ that occludes a vessel with an inner diameter of about 7 mm to about 10 mm may have a length of about 12 mm to about 14 mm and an expanded outer diameter of about 12 mm. Such an occlusive device 10′ may have an unexpanded outer diameter of about 0.035 inch (about 0.89 mm) and include as few as 20 struts 36′ and as many as 80 struts 36′ around its circumference.

In some embodiments, an occlusive device 10, 10′, 10″, 10″′, 10″″, etc. (hereinafter referred to as occlusive device 10 for the sake of simplicity) according to this disclosure may include a coating (e.g., an expandable coating, a resiliently expandable/compressible coating, etc.) and/or a filler (e.g. an expandable coating, a resiliently expandable/compressible fill material). A coating and/or a filler may provide for even further occlusion. A coating may extend over an outer surface of the body 12, 12′, 12″, 12″′, 12″″, etc. (hereinafter referred to as body 12 for the sake of simplicity) of the occlusive device 10. A filler may be confined with a lumen of the hypotube that defines the body 12 of the occlusive device 10. The filler may absorb blood, which may enhance the ability of the occlusive device 10 to stop, or occlude, the flow of blood and substances carried by the blood through the occluded vessel. In some embodiments, the coating and/or filler may be bonded to the body 12.

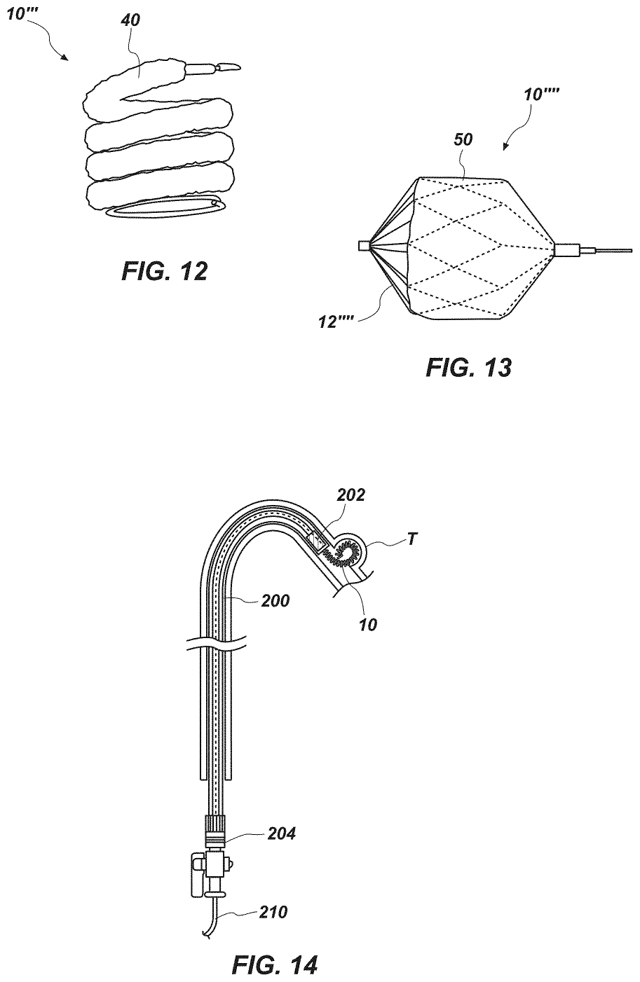

As an example, a coating and/or filler may comprise an expandable hydrogel, which may swell once the occlusive device 10 is placed to improve filling volume and packing density. As another example, depicted by FIG. 12, an occlusive device 10″′ may be provided with an expandable polymer foam or mesh 40, which may be formed from a shape memory polymer (SMP), such as a polyurethane SMP (e.g., N,N,N′,N′-Tetrakis (2-hydroxypropyl) ethylenediamine (HPED); 2,2′,2″-nitrilotriethanol (TEA); 1,6-diisocyanatohexan (HDI); trimethylhexamethylene diisocyanate (2,2,4- and 2,4,4-mixture) (TMHDI), etc.). In another example, the occlusive device 10 may be provided with flexible filaments or fibers, which may be located within a lumen of the body 12, extend through slits 32 (FIGS. 5B-5D), and/or be provided on an outer surface of the body 12. As yet another example, depicted by FIG. 13, a fabric (e.g., PTFE, etc.) or a film 50 (e.g., a polymer film, etc.) may cover at least a portion of the body 12″″ of the occlusive device 10″″ (e.g., an outer surface and/or an inner surface thereof, etc.) in a manner that prevents fluid flow through open slits 32″″ (FIGS. 5B-5D) in the body 12″″. Other embodiments of fillers include, without limitation, other polymers, calcium alginate, starch, and other solid or semi-solid materials.

A filler may be introduced into the occlusive device 10 during its manufacture. In embodiments where the occlusive device 10 includes ends 16′ and 17′ that are tapered or pinched (see, e.g., FIGS. 8C, 8D, and 8E) and/or smaller cells 33′ (see, e.g., FIGS. 9D), the ends 16′, 17′ and/or small cells 33′ may enable the occlusive device 10′ to receive and at least temporarily hold the filler(s).

In addition or as an alternative to enhancing the ability of an occlusive device 10 to occlude, a coating and/or a filler may impart the occlusive device 10 with further properties. For example, a coating and/or filler may absorb fluids from the body of the subject, which may promote embolization.

In another example, a filler may impart an occlusive device 10 with radio opacity. Such a filler may be bonded to the body 12 in a manner that enables the filler to expand with and/or inside of the body 12 and that prevents the filler from migrating out of the body 12 when in its expanded state. Such a filler may comprise cotton, nylon, fiber, filament, and/or another suitable material. The filler may be absorbent. In some embodiments, the filler may be manufactured with a radiopaque material (e.g., tungsten, barium, iodine, bismuth trioxide (bismuth (III) oxide and/or Bi₂O₃), etc.) and/or another material that facilitates x-ray visualization.

The filler may carry (e.g., absorb, etc.) a substance that is to be delivered to a target site within a body of a subject. Examples of substances that may be carried by the filler include, without limitation, contrast media, drugs, a treatment (e.g., a radioactive material for radiation therapy, etc.), and the like, which may be applied to the filler during manufacture or tableside by a clinician during a procedure prior to deployment, during deployment, or after deployment.

A clinician may inject a substance into a shipping and storage tube containing the occlusive device 10 prior to loading the occlusive device 10 into a catheter for delivery into a subject's body. Any filler in the occlusive device 10 may absorb or otherwise carry the substance. Alternatively, such a substance may be applied to and absorbed by a filler during manufacture of the occlusive device 10.

In embodiments where the substance comprises contrast media, the contrast media will be radiopaque under fluoroscopic x-ray to guide in placement as the occlusive device 10 is pushed through the catheter to the target location and while the occlusive device 10 is deployed at the target location. After deployment, the contrast media may dissipate, elute, and/or wash out of the occlusive device 10. This allows the occlusive device 10 to be seen during placement, but decrease in x-ray visualization after placement, which can be advantageous to viewing adjacent anatomy and pathology. In embodiments where the substance comprises a drug, a treatment (e.g., an oncolytic, radioactive isotope, such as yttrium-90 (Y90), during a radioembolization procedure, etc.), a nutrient, a diagnostic reagent, a marker, a targeting compound, or the like, the substance may be eluted once the occlusive device 10 is placed at the target location within the body of a subject. The rate at which the substance elutes from the filler over time and, thus, from the occlusive device 10 may depend at least partially upon any of a variety of factors that should be apparent to one of ordinary skill in the relevant art, including, but not limited to the composition, density, and/or volume of the filler. Elution may occur over a duration of minutes, hours, days, or even longer periods of time.

Optionally, a clinician may deploy the occlusive device 10 (with or without a filler) and inject a substance into the catheter that delivers the occlusive device 10 before or while the occlusive device is advanced along the catheter. This may allow clinicians to inject the substance into the catheter while the constrained occlusive device 10 is pushed through the catheter, but enable the substance to dissipate after the occlusive device 10 has been placed at its target location.

As another option, a substance may be introduced through the catheter and into the occlusive device 10 or a filler thereof after the occlusive device 10 has been deployed.

A substance may also be applied directly to the body 12 of the occlusive device 10 (e.g., to one or more struts 36 thereof, etc.). The substance may be bonded to, painted on, adhered to, or otherwise applied to the body 12 of the occlusive device 10. As another option, bands that carry the substance (e.g., radiopaque bands, etc.) may be crimped onto one or more struts 36 and/or one or both ends 16, 17 of the body 12.

In some embodiments, an occlusive device 10 may include a sensor. The sensor may comprise a passive sensor or an active sensor. The sensor may be located within or secured to the body 12 of the occlusive device 10. In some embodiments, the sensor may comprise a radiofrequency identification sensor, or chip.

A method of manufacturing an occlusive device 10 may employ a hypotube (e.g., a 0.035 inch (0.89 mm) OD and ˜0.030 inch (˜0.76 mm) ID nitinol hypotube, etc.). Slits 32 (FIGS. 5B-5D) may be cut into the hypotube by any suitable process (e.g., by laser cutting, mechanically (e.g., by computer-numeric control (CNC) machining, etc.), by electrical discharge machining (EDM), by chemical etching, etc.). The slits 32 may be cut from end-to-end of the hypotube so the outer diameter of the hypotube expands consistently along the entire length of the hypotube. Alternatively, the slits 32 may not extend to locations of the hypotube (e.g., one or both ends thereof, one or more intermediate locations, etc.) that are not intended to expand, or remain constrained, when the occlusive device 10 is deployed. Constrained locations may be useful for a variety of purposes, such as retaining a material within an interior of the occlusive device 10, providing a connection point for a deployment mechanism that facilitates deployment and/or positioning of the occlusive device 10, or coupling the occlusive device 10 to another occlusive device.

Cutting of the slits 32 the hypotube may result in struts 36 with blunt edges or struts 36 with sharp edges. Additionally, cutting of the slits may include the definition of features along edges of the struts 36, such as teeth, serrations, edge roughness, or the like. Such features may enable the resulting occlusive device 10 to be secured in place in a target location within a subject's body, which may promote an endothelial and/or thrombotic response and/or otherwise prevent migration of the occlusive device 10 once it has been positioned in the target location.

Edges of the struts 36, which are defined by the slits 32, may be modified after the slits 32 have been cut. In some embodiments, the edges may be burnished. In other embodiments, the edges may be sharpened.

Other features may also be cut into the hypotube. For example, slots, holes, channels, or other features may be cut into one or both ends 16, 17 and/or one or more struts 36 of the hypotube. These features may engage with a deployment mechanism (e.g., a detachable pusher, etc.). In a specific embodiment, one or more round (e.g., 0.003 inch, or 0.076 mm, diameter, etc.) female indentations or channels may be formed in an end 16 of the hypotube; these female indentations or channels may receive extendable/retractable, round (e.g., 0.003 inch (0.076 mm) or smaller diameter) male features of a deployment mechanism. The connection may be secure enough for a user to push or pull the occlusive device 10 through a delivery device 200 (FIG. 14), such as a catheter, sheath, cannula, needle, or the like.

The cut hypotube may then be loaded onto a mandrel (e.g., a hard steel mandrel) of desired shape (e.g., tapered straight, helical, funnel, etc.). As the cut hypotube is loaded onto the mandrel, the hypotube may expand, increasing its ID and OD (e.g., to about 0.075 inch, or about 1.9 mm). Expansion of the cut hypotube may causes slits 32 of the hyptotube to open and struts 36 (FIGS. 5B-5D) of the hypotube to be exposed. The expanded, cut hypotube may then be heated to a sufficient temperature (e.g., about 400° C. to about 600° C., etc.) for a sufficient duration (e.g., up to 1 hour, etc.) to set the nitinol in its expanded state. The hypotube may then be cooled; it may remain on the mandrel or it may be removed from the mandrel. The cooled hypotube may then be constrained back to its original OD by physically squeezing the OD and/or pushing the expanded hypotube into a funneled hypotube fixture that funnels from 0.080 inch (about 2.0 mm) ID down to about 0.035 inch (about 0.89 mm) or smaller. The constrained hypotube may then be loaded (e.g., pushed, etc.) into a shipping and storage tube to keep the hypotube constrained until deployment. This manufacturing method applies to hypotubes of all sizes, including but not limited to 0.014 inch (0.36 mm) OD, 0.018 inch (0.46 mm) OD, 0.025 inch (0.64 mm) OD, 0.027 inch (0.69 mm) OD, and other ODs, IDs, and lengths.

Referring now to FIG. 14, a method of using an occlusive device 10 includes advancing a distal tip 202 of a delivery device 200, such as the depicted catheter, a sheath, a hypotube, a cannula, a needle, or the like, to a target location T within a body of a subject. The occlusive device 10 may be transferred from a loading device (not shown) into a proximal end 204 of the delivery device 200. The occlusive device 10 may be advanced along the length of the delivery device 200 until it reaches the distal tip 202. As the occlusive device 10 emerges, or is deployed from, from the distal tip 202, the occlusive device 10 may at least partially expand and may be positioned against a surface of the target location T (e.g., against the intima of a vessel, etc.). Deployment may be achieved by pushing the constrained occlusive device 10 (e.g., with a deployment mechanism 210, etc.) distally out of the distal tip 202 and/or pulling the delivery device 200 proximally while maintaining a position of the occlusive device 10 within the body of the subject (e.g., at the target location T, etc.).

Once the occlusive device 10 has been fully deployed from the distal tip 202, it may assume its final shape.

Once the occlusive device 10 exits the delivery device 200, the deployment mechanism 210 may remain connected to the occlusive device 10. This may allow the clinician to confirm placement accuracy. Optionally, the clinician may push, pull, drag, or otherwise move the at least partially expanded occlusive device 10 in a manner that positions the occlusive device 10 at the target location T (e.g., with a deployment mechanism 210, etc.). Such movement may also denude, agitate, or mechanically irritate the intima at the target location T to elicit an inflammatory response (with or out without injecting any sclerosant), which may promote temporary or permanent immobilization of the occlusive device 10 at the target location T and, thus, temporary or permanent embolization.

If the placement accuracy is acceptable, the deployment mechanism 210 may be uncoupled from the occlusive device 10 (e.g., by retracting the extendable/retractable round male features of the deployment mechanism 210 to detach the deployment mechanism 210 from the occlusive device 10.

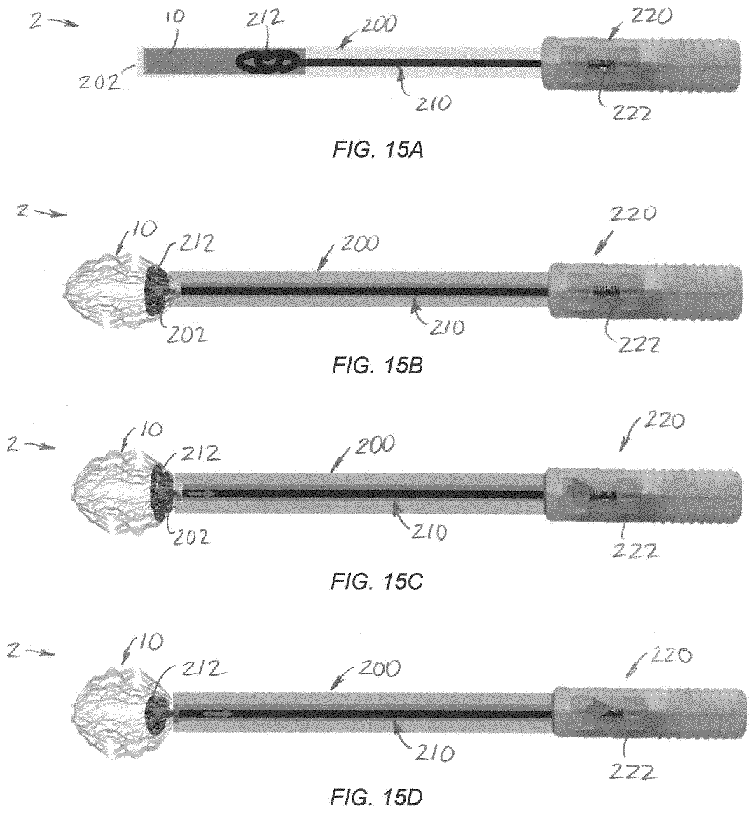

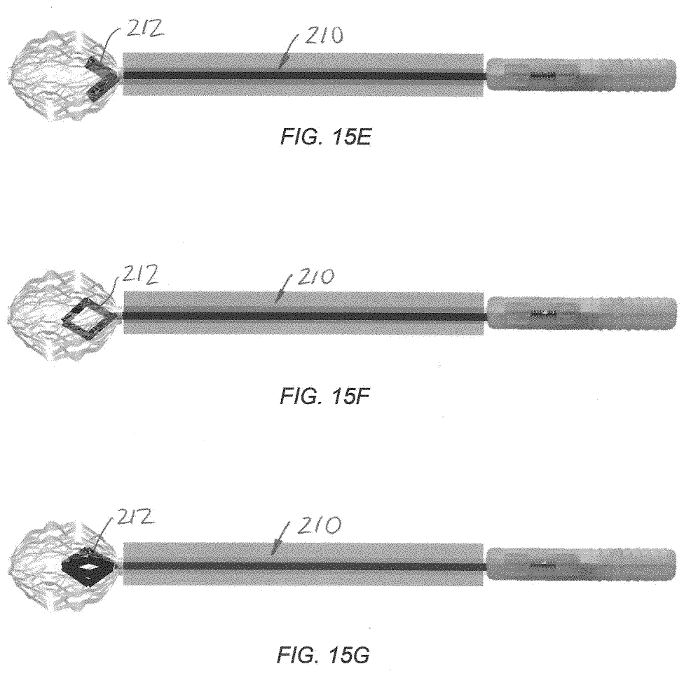

FIGS. 15A-15H depict an embodiment of a delivery system 2 that may be used to deliver an occlusive device 10 to a target location T (FIG. 14) within the body of a subject. As shown in FIG. 15A, the delivery system 2 includes a delivery device 200 (e.g., a catheter, a sheath, a hypotube, a cannula, a needle, etc.), a control wire 210, and, optionally, a slider 220 that controls distal and/or proximal movement of the control wire 210 through a lumen of the delivery device 200. In some embodiments, the delivery device 200 may comprise a hypotube formed from any suitable material (e.g., stainless steel, nitinol, polyester, PEEK, etc.) with an outer diameter of about 0.015 inch (about 0.38 mm) to about 0.035 inch (about 0.89 mm) or larger and an inner diameter large enough to accept the occlusion device 10 and the control wire 210. The control wire 210 may comprise a wire formed from any suitable material (e.g., stainless steel, nitinol, polyester, PEEK, etc.) and may have an outer diameter of about 0.006 inch (about 0.15 mm) to about 0.020 inch (about 0.51 mm). The slider 220, if any, may comprise a hand-held slider that may be held within a clinician's hand and include a switch 222 that may be moved with the clinician's thumb or finger on the hand that holds the slider 220 to control movement of the control wire 210 through the delivery device 200.

The control wire 210 may include a distal end 212 that carries the occlusive device 10 until the occlusive device 10 is delivered to the target location T. As shown in FIG. 15A, while the occlusive device 10 is present within a lumen of the delivery device 200 and the distal end 212 of the control wire 210 is present within the interior of the occlusive device, the delivery device 200 constrains the occlusive device 10 and the occlusive device 10 constrains the distal end 212 of the control wire 210. Thus, the distal end 212 of the control wire 210 abuts against an interior surface of the occlusive device 10, thereby engaging the occlusive device 10. The distal end 212 of the control wire 210 may remain relatively linear or curvilinear while the distal end 212 is constrained within the interior of the occlusive device 10. The occlusive device 10 is, in turn, constrained within the lumen of the delivery device 200. As depicted by FIG. 15B, the distal end 212 of the control wire 210 may expand (e.g., to an outer diameter of up to twice its native outer diameter, etc.) when the distal end 212 of the control wire 210 and the occlusive device 10 carried by the distal end 212 are advanced beyond the distal tip 202 of the delivery device 200.

Since, as shown in FIG. 15B, the distal end 212 of the control wire 210 may expand with and within the occlusive device 10 it carries once the occlusive device 10 and the distal end 212 of the control wire 210 advance beyond the distal tip 202 of the delivery device 10, the control wire 210 may continue to engage the occlusive device and, thus, be used to reposition the occlusive device 10 once the occlusive device 10 exits the delivery device 200.

A configuration of the distal end 212 of the control wire 210 may enable it to be re-constrained more easily than the occlusive device 10 can be re-constrained. Thus, once the occlusive device 10 has been properly positioned at its target location T (FIG. 14), as shown in FIGS. 15C and 15D, the distal end 212 of the control wire 210 and the occlusive device 10 may be pulled proximally against the distal tip 202 of the delivery device 200 and the distal end 212 of the control wire 210 may be withdrawn from the occlusive device 10 and into the distal tip 202 of the delivery device 200. The delivery device 200 and the control wire 210 may then be withdrawn from the body of the subject.

While FIGS. 15A-15D show the distal end 212 of the control wire 210 has having a coiled shape when the distal end 212 is unconstrained and, thus, expanded, distal ends 212 with different expanded sizes and distal ends 212 of different shapes are also within the scope of this disclosure. FIGS. 15E-15G depict a few different configurations of expanded distal ends 212.

Without limitation, an occlusive device 10 according to this disclosure may be used to facilitate luminal filling, decrease flow, improve thrombosis, improve hyperplasia, lower x-ray density, or otherwise promote occlusion. Such an occlusive device 10 may be used in connection with a variety of conditions, including, without limitation, arteriovenous malformations, bleeds, perforations, aneurysms, fibroids, varices, congestion, distal emboli, and other conditions. The occlusive device 10 may be used to treat COVID-19 patients who present with increased D-dimer levels (fibrin protein antigen fragments found in blood test indicated clotting disorder) and life-threatening blood clots in the heart, lungs, brain, and peripheral vessels. Bleeding is a complication of blood clots and bleeding may be treated with embolic devices. See, e.g.:
https://www.ncbi.nlm.nih.gov/pmc/articles/PMC7203058/
https://www.medicalnewstoday.com/articles/covid-19-ive-never-seen-such-sticky-blood-says-thrombosis-expert
https://www.sciencedaily.com/releases/2020/06/20060125129.htm
https://pubmed.ncbi.nlm.nih,gov/32339221/
https://www.ncbi.nlm.nih.gov/pmc/articles/PMC7146714/
https://pubmed.ncbi.nlm.nih.gov/32316063/
https://www.ncbi.nlm.nih.gov/pmc/articles/PMC7225095/
https://www/ncbi.nlm.nih.gov/pmc/articles/PMC7229939/
https://www.ncbi.nlm.nih.gov/pmc/articles/PMC7255402/
https://www.ncbi.nlm.nih.gov/pmc/articles/PMC7177070/,
   the disclosures of which are hereby incorporated herein.

Although the preceding disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. An occlusive device, comprising:
a hypotube with struts that rotate and move apart from each other upon expansion of the hypotube to an increased outer diameter and a final shape upon deployment to a target location within a body of a subject, the increased outer diameter and the final shape capable of at least partially occluding a passage through a portion of the body of the subject.

2. The occlusive device of claim 1, wherein ends of the hypotube are oriented along a longitudinal axis of the final shape of the hypotube.

3. The occlusive device of claim 1, wherein, with the hypotube in the final shape, ends of the hypotube are tapered from an expanded outer diameter of the hypotube to a terminal outer diameter that is smaller than the expanded outer diameter.

4. The occlusive device of claim 1, wherein, with the hypotube in the final shape, portions of the hypotube adjacent to ends of the hypotube taper from an expanded outer diameter of the hypotube to a pinched outer diameter that is smaller than the expanded outer diameter, the ends of the hypotube comprising flares with a terminal outer diameter that exceeds the pinched outer diameter.

5. The occlusive device of claim 4, wherein the flared outer diameter is at least as large as the expanded outer diameter.

6. The occlusive device of claim 1, further comprising:
a filler within an interior of the hypotube.

7. The occlusive device of claim 6, wherein the filler comprises an absorbent material.

8. The occlusive device of claim 7, wherein the filler further comprises another substance.

9. The occlusive device of claim 8, wherein the another substance, when present within the body of the subject, elutes from the absorbent material over time.

10. The occlusive device of claim 8, wherein the another substance comprise at least one of a contrast medium, a drug, and a treatment.

11. The occlusive device of claim 1, wherein a first portion of the hypotube includes first struts that move a first distance apart from each other upon expansion of the hypotube and a second portion of the hypotube includes second struts that move a second distance apart from each other upon expansion of the hypotube, the first distance being greater than the second distance.

12. The occlusive device of claim 1, wherein the final shape is a coiled shape.

13. The occlusive device of claim 1, wherein the final shape is a somewhat spherical shape.

14. The occlusive device of claim 1, wherein the final shape has a length of at least 6 mm and an outer diameter of at least 6 mm.

15. A delivery system for an occlusive device, comprising:
a delivery device with a lumen; and
a control wire positionable within the lumen of the delivery device and including a distal end that has:
a constrained state in which the distal end engages the occlusive device while constrained within the lumen of the delivery device to facilitate movement of the occlusive device along at least a portion of a length of the lumen; and
an expanded state that engages the occlusive device while expanded outside of the lumen of the delivery device.

16. The delivery system of claim 15, further comprising:
a slider associated with the delivery device and the control wire in a manner that facilitates movement of the control wire distally and proximally through the lumen of the delivery device.

17. A method of occluding a passage or a void within a body of a subject, comprising:
introducing an occlusive device to a target location within the body of the subject in a constrained state, including:
introducing a delivery device in which the occlusive device and a portion of a control wire reside into the body of the subject;
advancing a distal tip of the delivery to the target location;
advancing the control wire distally through the delivery device, a distal end of the control wire advancing the occlusive device distally, until the occlusive device exits the distal tip of the delivery device;
as the occlusive device is introduced to the target location:
expanding an outer diameter of a hypotube of the occlusive device, placing the occlusive device in an expanded state; and
allowing the occlusive device to assume a final shape; and
once the occlusive device has expanded and resides at the target location:
withdrawing the control wire proximally through the delivery device, including withdrawing the distal end of the control wire from the interior of the occlusive device; and
withdrawing the delivery device from the body of the subject.

18. The method of claim 17, wherein introducing the occlusive device to the target location includes:
providing the occlusive device in a lumen of the delivery device; and
providing the control wire within the lumen of the delivery device with the distal end of the control wire residing within the interior of the occlusive device.

19. The method of claim 17, further including, as the occlusive device is introduced to the target location:
expanding the distal end of the control wire as the portion of the occlusive device within which the distal end resides exits the distal tip of the delivery device.

20. The method of claim 17, withdrawing the control wire proximally through the delivery device includes pulling the occlusive device in the expanded state against the distal tip of the delivery device and withdrawing the distal end of the control wire from the interior of the occlusive device comprises continuing to pull the control wire proximally from the interior of the occlusive device and into the lumen of the delivery device.
